(19) United States
(12) Patent Application Publication
(10) Pub. No.: US 2024/0336662 A1
(43) Pub. Date: Oct. 10, 2024

(54) COMPOSITIONS AND RELATED METHODS FOR MODULATING ALKALOID PRODUCTION BY CONTROLLING PMT PROMOTER ACTIVATION MEDIATED BY TRANSCRIPTIONAL FACTORS ERF AND MYC

(71) Applicant: University of Virginia Patent Foundation, Charlottesville, VA (US)

(72) Inventors: Michael Paul TIMKO, Charlottesville, VA (US); Paul J. RUSHTON, Brookings, SD (US); Sheng-Cheng HAN, Charlottesville, VA (US); Hongbo ZHANG, Chongging (CN); Marta Tatiana BOKOWIEC, Charlottesville, VA (US)

(73) Assignee: University of Virginia Patent Foundation, Charlottesville, VA (US)

(21) Appl. No.: 18/754,305

(22) Filed: Jun. 26, 2024

Related U.S. Application Data
(63) Continuation of application No. 18/363,120, filed on Aug. 1, 2023, now Pat. No. 12,054,523.
Application No. 18/363,120 is a continuation of application No. 17/517,305, filed on Nov. 2, 2021, now Pat. No. 11,760,781.
Application No. 17/517,305 is a continuation of application No. 16/369,614, filed on Mar. 29, 2019, now Pat. No. 11,168,118.
Application No. 16/369,614 is a continuation of application No. 15/644,199, filed on Jul. 7, 2017, now Pat. No. 10,280,203.
Application No. 15/644,199 is a division of application No. 14/311,684, filed on Jun. 23, 2014, now Pat. No. 9,701,978.
Application No. 14/311,684 is a continuation of application No. 12/676,871, filed on Nov. 10, 2010, now Pat. No. 8,759,101.
(60) Provisional application No. 60/935,948, filed on Sep. 7, 2007.
(60) Provisional application No. 60/935,947, filed on Sep. 7, 2007.

(51) Int. Cl. C07K 14/415; C12N 15/82

(52) U.S. Cl. CPC C07K 14/415; C12N 15/8243

(57) ABSTRACT

Compositions and methods for modifying the production levels of alkaloids in plants are provided. Alkaloid production can be genetically controlled by modulating the transcriptional activation of PMT genes mediated by members of the ERF family and/or Myc family of transcription factors. Novel nucleotide sequences encoding the Myc family of transcription factors are also provided.

### BACKGROUND

Nicotine production from polyamine putrescine, a precursor of nicotine, can be produced by two pathways in plants. Putrescine can be synthesized directly from ornithine in a reaction catalyzed by the enzyme ornithine decarboxylase, or can be produced indirectly from arginine in a sequence of reactions initiated by arginine decarboxylase. The first committed step in nicotine biosynthesis is the conversion of putrescine to N-methylputrescine by putrescine N-methyltransferase (“PMT”). N-methylputrescine is subsequently oxidized by a diamine oxidase, and is cyclized to produce a 1-methyl-Δ¹-pyrrolium cation, which is subsequently condensed with nicotinic acid to produce nicotine.

There is a need for compositions and improved methods for genetically regulating the production levels of nicotine and other alkaloids in plants, including transgenic plants, transgenic tobacco plants, recombinant stable cell lines, recombinant stable tobacco cell lines, and derivatives thereof.

### SUMMARY

In various embodiments, compositions and methods for modifying the production levels of nicotine and other alkaloids in plants are provided. Nicotine and other alkaloid production can be genetically modified by modulating the transcriptional activation of PMT genes mediated by members of the ERF family and/or Myc family of transcriptional factors.

### BRIEF DESCRIPTION OF DRAWINGS

FIG. 1 illustrates multiple signal transduction pathways, induced by various stimuli that regulate PMT promoter activation/repression in plants.

FIGS. 2A-2C show the identification of transcription factors of the ERF family that can bind specifically to the GAG motif.

FIGS. 3A-3C show relative expression levels of transcripts for NtERF2, NERF3, and NERF5, measured by RT-PCR in BY-2 cell extracts. The expression levels of NtERF2 and NtERF5 were responsive to MeJA-inducible pathway. The expression level of NtERF3 was responsive to ethylene (or ethephon) inducible pathway.

FIG. 4 shows that NtERF5 can bind to the GAG motif by in vitro gel mobility shift assay.

FIGS. 5A-5C show that NtERF5 can activate the NtPMT1a promoter by binding to the AT-rich region of the GAG motif.

FIG. 6A shows that NtERF5 over-expression results in GUS reporter expression in a MeJA-dependent manner by binding to 4xGAG motifs positioned upstream of a promoter that drives GUS reporter expression. Overexpressed cell lines (“OE”) that have been stably integrated with 4XGAG::GUS reporter were transiently transfected with expression vectors over-expressing NtERF5.

FIG. 6B shows that NtERF5 over-expression is not sufficient to activate NtPMT expression in the absence of MeJA. NtPMT transcripts were detected in extracts of cell lines that were induced by MeJA.

FIG. 7 shows that NtERF2 over-expression activates NtPMT expression in a MeJA-independent manner.

FIGS. 8A-8C show that Myc3/Myc4 over-expression in BY-2 cells is inducible by MeJA.

FIG. 8D shows that Myc3/Myc4 over-expression in the roots of transgenic tobacco is inducible by MeJA.

FIG. 9 shows that Myc3 binds the G-box of the GAG motif by in vitro mobility shift assay.

FIG. 10 shows differential localization of Myc3 and Myc4, respectively, in BY-2 cells after MeJA exposure. NtMyc3-GFP localized to the nucleus after MeJA exposure. In contrast, NtMyc4-GFP was excluded from the nucleus after MeJA exposure.

FIG. 11 shows the polynucleotide sequence for NtMyc3 cDNA (SEQ ID NO:1).

FIG. 12 shows the polypeptide sequence for NtMyc3 protein (SEQ ID NO:2).

FIG. 13 shows the polynucleotide sequence for NtMyc4 cDNA (SEQ ID NO:3).

FIG. 14 shows the polypeptide sequence for NtMyc4 protein (SEQ ID NO:4).

FIG. 15 shows the polynucleotide sequence for NtERF2 cDNA (SEQ ID NO:5).

FIG. 16 shows the polynucleotide sequence for NtERF5 cDNA (SEQ ID NO:6).

### DETAILED DESCRIPTION

Regulation of nicotine biosynthesis is desirable in a variety of plants, especially in tobacco plants. Nicotine biosynthesis can be regulated by controlling the expression levels and/or the activities of enzymes involved in the nicotine biosynthetic pathway. In particular, an effective way to genetically regulate nicotine production is by controlling the transcriptional activation of promoters that control the expression of genes encoding putrescine N-methyltransferases (“PMT”). PMT is one of several critical enzymes involved in the nicotine biosynthetic pathway in plants, including tobacco plants. Various compositions and methods for modifying PMT expression levels in plants are provided, as further described below.

The production levels of nicotine, other alkaloids, and secondary metabolites can be genetically regulated by controlling the expression level of PMT, which correlates directly with their production levels. This can be accomplished by controlling PMT promoter activation that correlates with the expression levels of the PMT structural gene. PMT RNA transcripts produced by transcriptional processes are subsequently translated into PMT polypeptides that exhibit PMT enzymatic activity. The activation of the PMT promoter by sequence-specific transcriptional factors (“transcriptional activators”) can increase the levels of PMT RNA transcripts and PMT polypeptides produced. In contrast, the repression of the PMT promoter by sequence-specific transcriptional factors (“transcriptional repressors”) can decrease the levels of PMT RNA transcripts and PMT polypeptides produced.

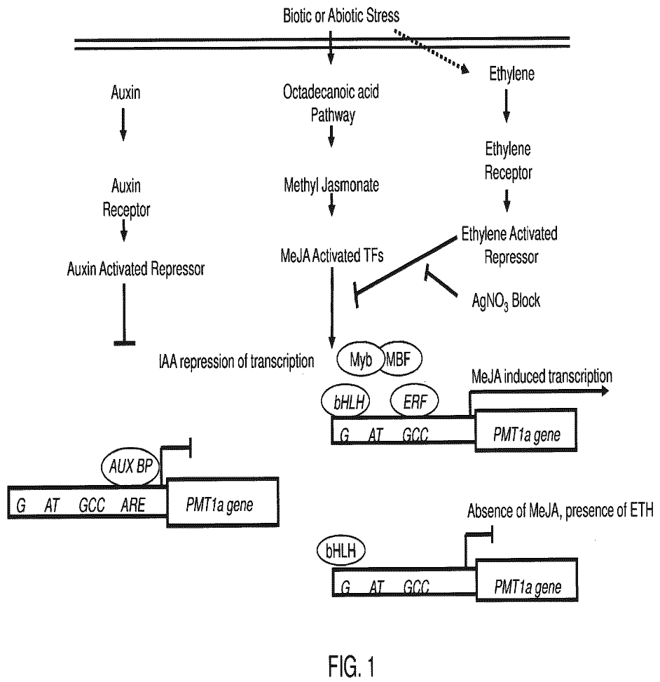

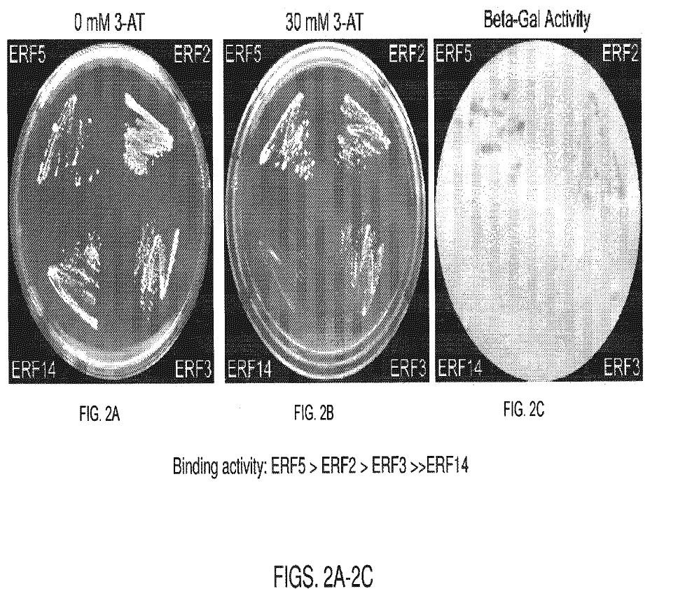

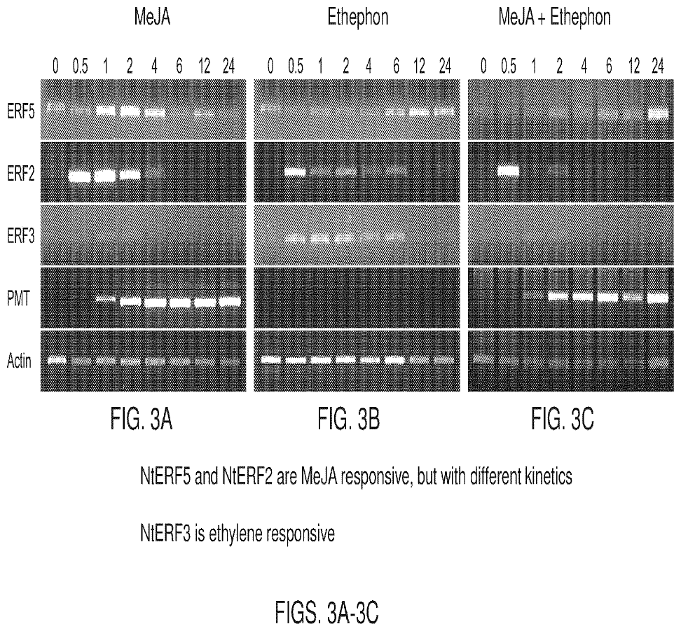

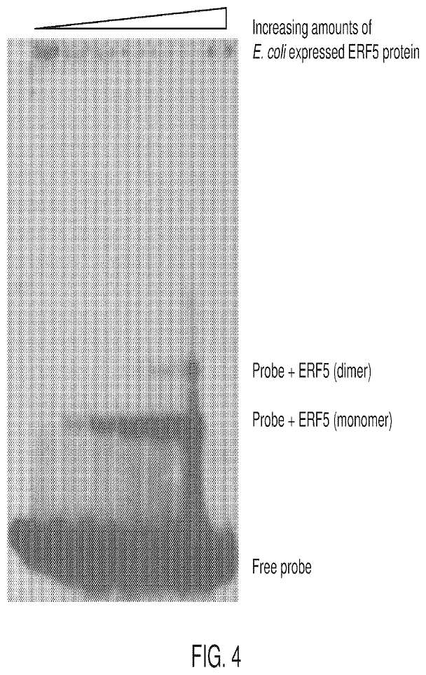

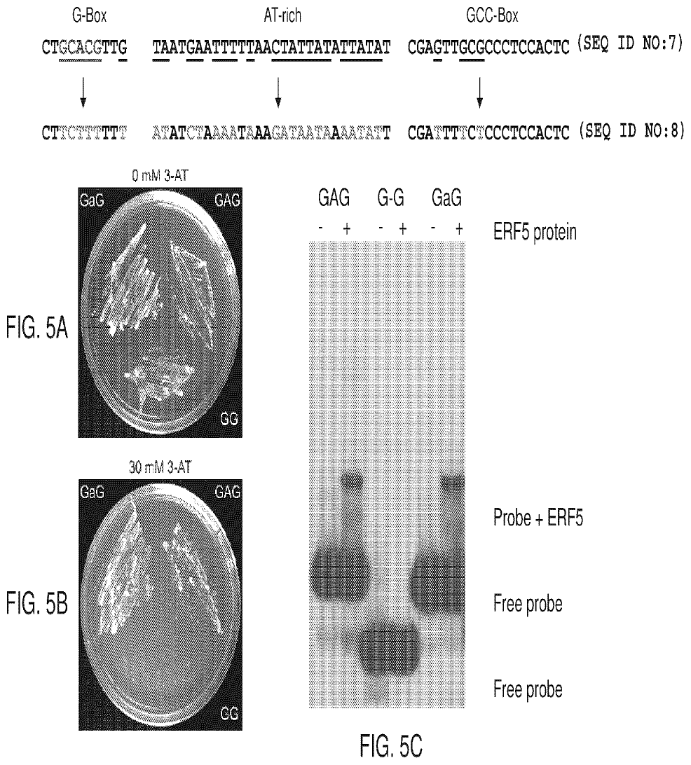

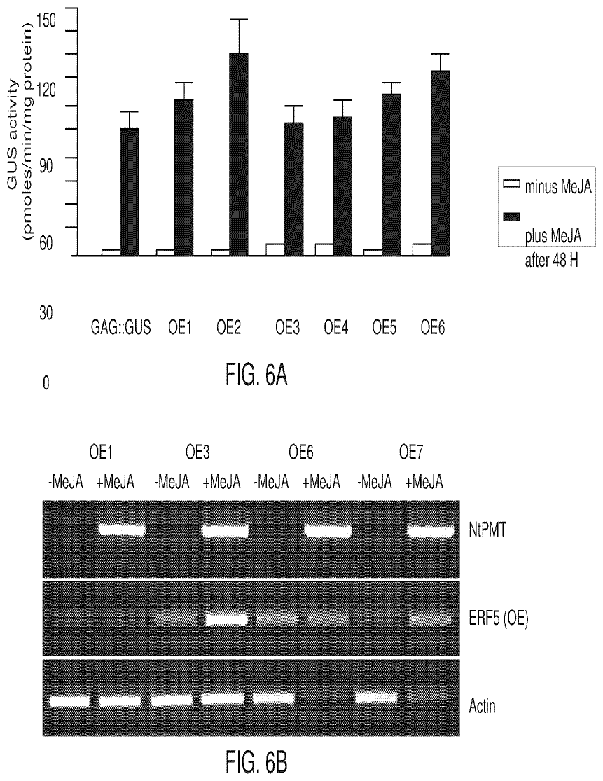

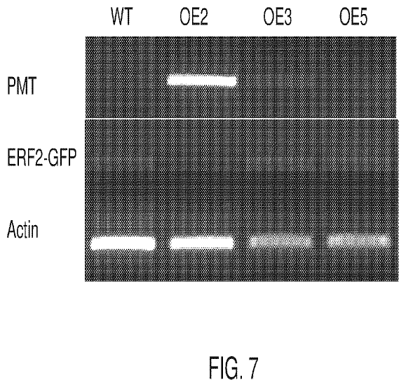

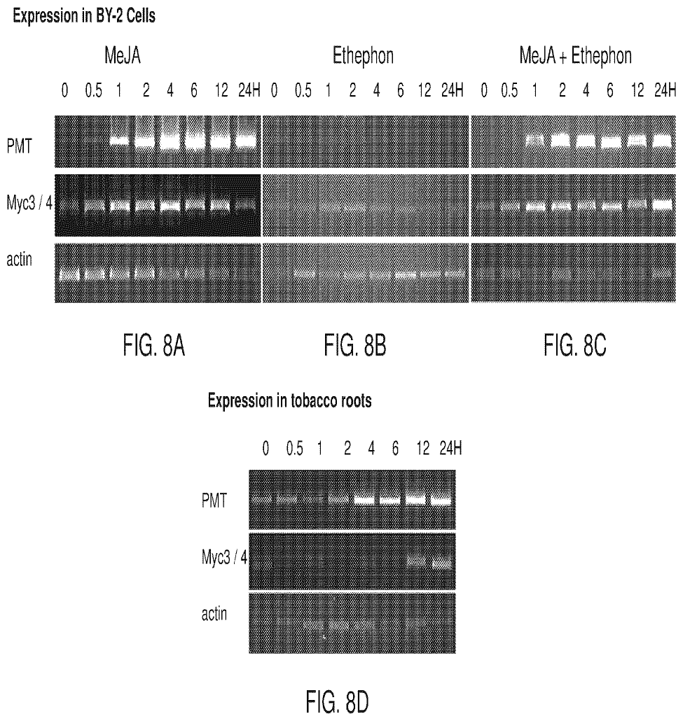

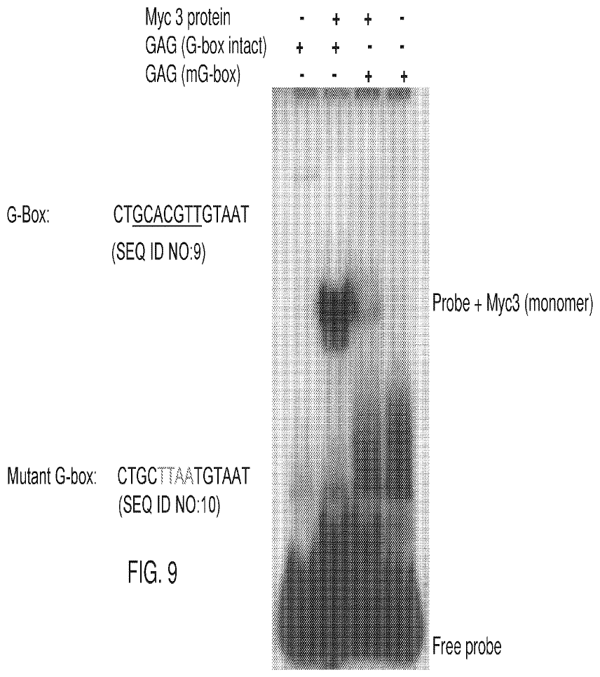

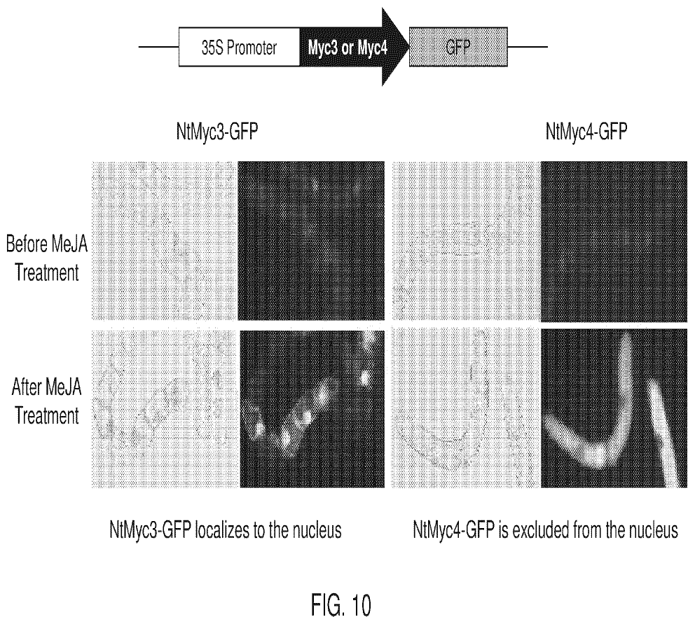

PMT promoter activation is responsive to various endogenous and exogenous signals, including phytohormones, wounding, and invasion by pathogens or insects. FIG. 1 illustrates the existence of multiple signal transduction pathways inducible by various phytohormones, including jasmonic acid (“JA”), auxin, and ethylene, which can affect PMT promoter activation. As shown in FIG. 1, the transcriptional regulation of PMT genes can be responsive to multiple signal transduction pathways that can be co-induced if multiple stimulants exist simultaneously in an environment. When a particular signal transduction pathway is induced, the expression level and/or the transcriptional activity of a transcriptional factor can be increased. Alternatively, the expression level and/or the transcriptional activity of a transcriptional factor can be decreased by inducing a particular signal transduction pathway. For example, a JA-inducible signal transduction pathway can be blocked by co-exposure to sufficient concentrations of auxin and/or ethylene that appear to have antagonistic effects on the JA-inducible pathway. In particular, PMT promoter activation induced by JA exposure can be blocked by exposure to auxin and/or ethylene.

In various plants, such as tobacco, the phytohormone jasmonic acid (“JA”) (3-oxo-2-(cis-2-pentenyl)-1-cyclopentaneacetic acid) and/or its methylated ester derivative (“MeJA”) can be utilized for inducing nicotine production. JA and/or MeJA can be utilized for inducing the expression of genes involved in the biosynthesis of secondary metabolites such as alkaloids, flavonoids, and terpenoids. Furthermore, JA and/or MeJA can be utilized to control various processes that regulate plant growth and environmental adaptation, including seed germination, regulation of carbon and nitrogen storage, photosynthesis, senescence, pollen development, fruit ripening, wound responses, and resistance to insects and pathogens. For regulating defense responses, JA and/or MeJA can be utilized to act synergistically with, or antagonistically against, two other plant hormones, salicylic acid (SA) and ethylene. JA and/or MeJA can be utilized for inducing the expression of genes encoding proteinase inhibitors involved in pest resistance and genes encoding defensins that exhibit antimicrobial activity. Therefore, various wound-induced and stress-induced biological responses of interest can be elicited by controlling the amount and extent of exposure to such phytohormones.

In Nicotiana tabacum, at least five PMT genes have been characterized: NtPMT1a, NtPMT1b, NtPMT2, NtPMT3, and NtPMT4. PMT gene expression in the roots of N. tabacum can be up-regulated by various stimuli, including topping procedures, the physical invasion by insects and/or other herbivores, and JA or MeJA exposure.

This disclosure describes a regulatory region upstream of the core promoter elements of PMT promoters, referred to as the “GAG motif,” that confers PMT promoters with responsiveness to JA and MeJA. The GAG motif comprises a G-box like element, an AT-rich element, and a GCC-like box element. The GAG motif functions optimally as an intact, tripartite unit, in that the three elements must be utilized together, and must be arranged so that the G-box like element is positioned upstream of the AT-rich element, which is positioned upstream of the GCC-like box element.

Based on experimental studies described herein, the GAG and the G-G derivative fragment (similar to the structure of the GAG motif but deficient in the AT-rich element) have been shown to be responsive to JA and MeJA when plants are exposed to these phytohormones. The JA and MeJA responsiveness of the GAG motif can be mediated by members of the ERF and Myc families of transcriptional factors, as described herein. The GAG motif confers the recruitment of ERF and Myc transcriptional factors and other transcriptional factors when operably-linked to a promoter of interest, which is operably-linked to a transgene of interest positioned downstream of the promoter of interest. Suitable transgenes include genes that encode various enzymes involved in the biosynthesis of alkaloids, nicotine, and flavonoids, for example.

The sub-elements of the GAG motif that can recruit particular members of the ERF and Myc families have been further characterized by results described herein. For example, the experimental results show that ERF2/ERF3/ERF5 are recruited to the GAG motif in a JA-inducible manner. This occurs through the GCC-like box element and requires some or all of the AT-rich element for recruitment. For example, the experimental results show that the G-box element can recruit NtMyc3/NtMyc4 in a JA-inducible manner.

For up-regulating or down-regulating various promoters of interest, the following compositions and methods are contemplated:

In general, various embodiments are directed to expression vectors that enable the over-expression of transcriptional factors, NtMyc3, NtMyc4, NtERF2, and/or NtERF5, for modulating the production levels of nicotine, other alkaloids, including various flavonoids. These expression vectors can be transiently introduced into host plant cells or stably integrated into the genomes of host plant cells to generate transgenic plants by various methods known to persons skilled in the art. When these expression vectors are stably integrated into the genomes of host plant cells to generate stable cell lines or transgenic plants, the over-expression of transcriptional factors, NtMyc3, NtMyc4, NERF2, and/or NtERF5, can be utilized as a method for modulating the promoter activation of endogenous promoters that are responsive to these transcriptional factors. Furthermore, such host plant cells can be further manipulated to receive heterologous promoter constructs that are responsive to transcriptional factors, NtMyc3, NtMyc4, NtERF2, and/or NtERF5. Furthermore, such host plant cells can be further manipulated to receive heterologous promoter constructs that have been modified by incorporating one or more GAG motifs upstream of the core elements of the heterologous promoter of interest.

Any promoter of interest can be manipulated to be responsive to JA and MeJA by incorporating one or more GAG motifs and/or derivative GAG motifs upstream of the promoter of interest. Suitable promoters include various promoters of any origin that can be activated by the transcriptional machinery of plant cells, such as various homologous or heterologous plant promoters and various promoters derived from plant pathogens, including bacteria and viruses. Suitable promoters include constitutively active promoters and inducible promoters.

For various expression vectors described below, various genes that encode enzymes involved in biosynthetic pathways for the production of alkaloids, flavonoids, and nicotine can be suitable as transgenes that can be operably-linked to a promoter of interest.

In another embodiment, an expression vector comprises a promoter operably-linked to the cDNA encoding Myc3, Myc4, ERF2, and/or ERF5. In another embodiment, a plant cell line comprises an expression vector comprising a promoter operably-linked to the cDNA encoding Myc3, Myc4, ERF2, and/or ERF5. In another embodiment, a transgenic plant comprises an expression vector comprising a promoter operably-linked to the cDNA encoding Myc3, Myc4, ERF2, and/or ERF5. In another embodiment, methods for genetically modulating the production of alkaloids, flavonoids, and nicotine are provided, comprising: introducing an expression vector comprising a promoter operably-linked to the cDNA encoding Myc3, Myc4, ERF2, and/or ERF5.

In another embodiment, an expression vector comprises a first promoter operably-linked to cDNA encoding Myc3, Myc4, ERF2, and/or ERF5; and a second promoter operably-linked to cDNA encoding an enzyme involved in the biosynthesis of alkaloids. In another embodiment, a plant cell line comprises an expression vector comprising a first promoter operably-linked to cDNA encoding Myc3, Myc4, ERF2, and/or ERF5; and a second promoter operably-linked to cDNA encoding an enzyme involved in the biosynthesis of alkaloids. In another embodiment, a transgenic plant comprises an expression vector comprising a first promoter operably-linked to cDNA encoding Myc3, Myc4, ERF2, and/or ERF5; and a second promoter operably-linked to cDNA encoding an enzyme involved in the biosynthesis of alkaloids. In another embodiment, methods for genetically modulating the production level of alkaloids are provided, comprising: introducing an expression vector comprising a first promoter operably-linked to cDNA encoding Myc3, Myc4, ERF2, and/or ERF5; and a second promoter operably-linked to cDNA encoding an enzyme involved in the biosynthesis of alkaloids.

In another embodiment, an expression vector comprises a first promoter operably-linked to cDNA encoding Myc3, Myc4, ERF2, and/or ERF5; and a second promoter operably-linked to cDNA encoding an enzyme involved in the biosynthesis of flavonoids. In another embodiment, a plant cell line comprises an expression vector comprising a first promoter operably-linked to cDNA encoding Myc3, Myc4, ERF2, and/or ERF5; and a second promoter operably-linked to cDNA encoding an enzyme involved in the biosynthesis of flavonoids. In another embodiment, a transgenic plant comprises an expression vector comprising a first promoter operably-linked to cDNA encoding Myc3, Myc4, ERF2, and/or ERF5; and a second promoter operably-linked to cDNA encoding an enzyme involved in the biosynthesis of flavonoids. In another embodiment, methods for modulating the production level of flavonoids are provided, comprising: introducing an expression vector comprising a first promoter operably-linked to cDNA encoding Myc3, Myc4, ERF2, and/or ERF5; and a second promoter operably-linked to cDNA encoding an enzyme involved in the biosynthesis of flavonoids.

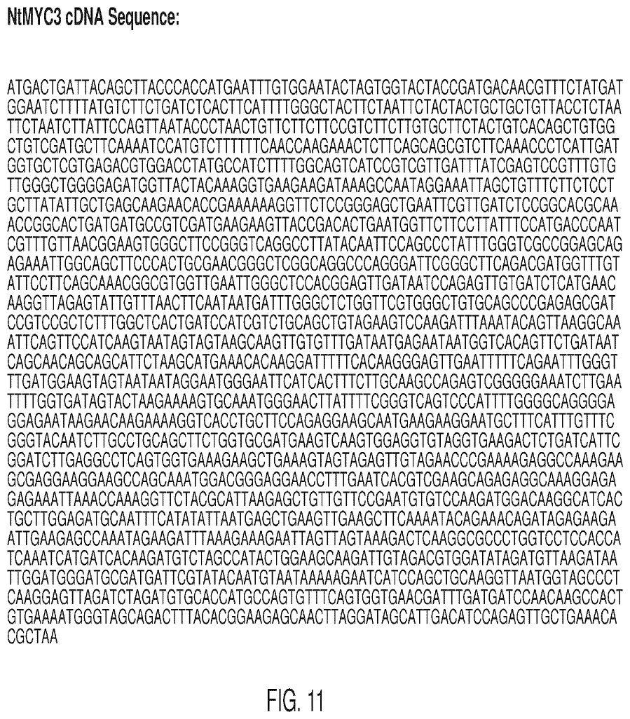

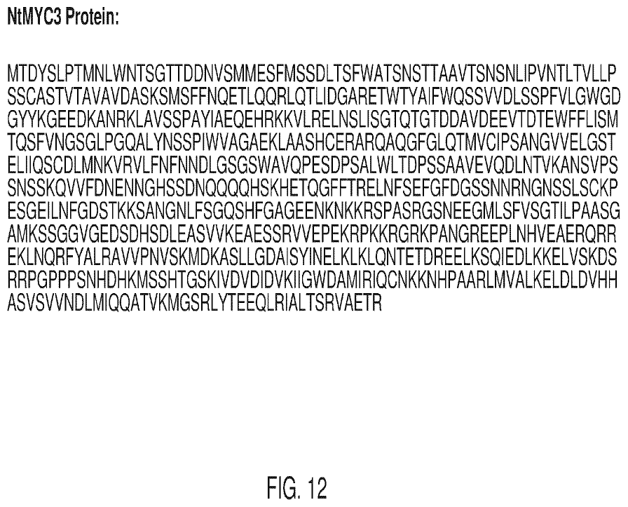

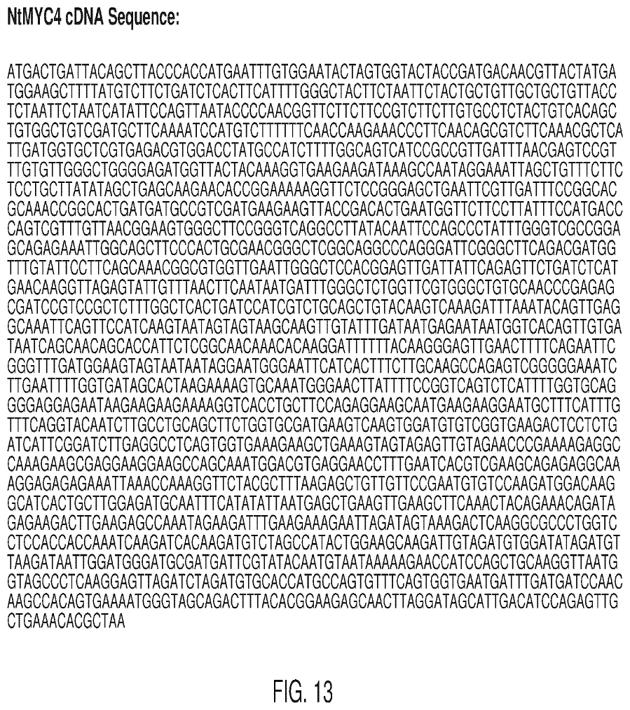

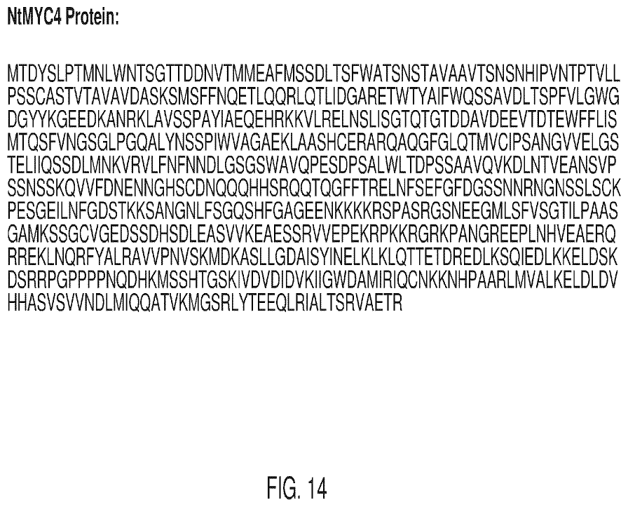

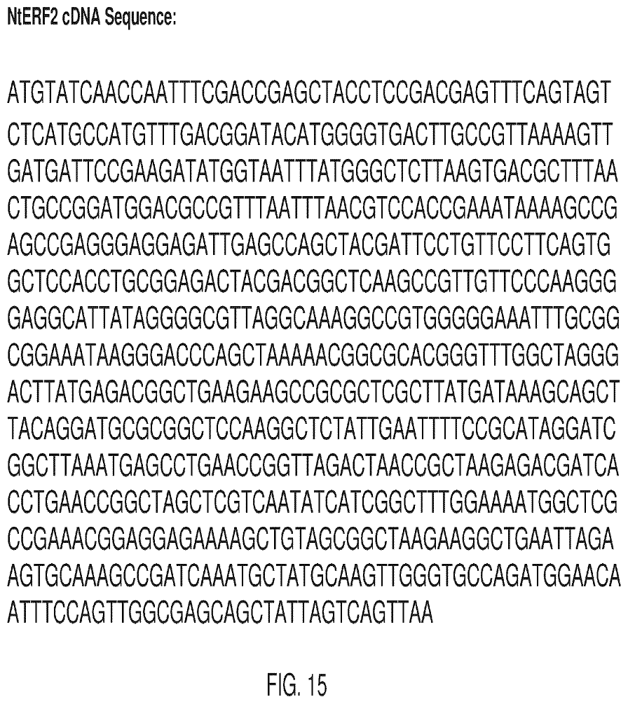

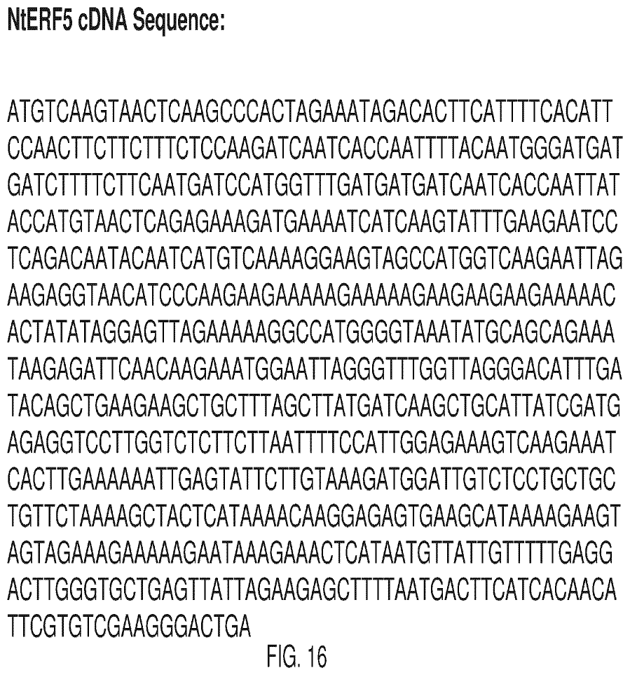

In another embodiment, an expression vector comprises a first promoter operably-linked to cDNA encoding Myc3, Myc4, ERF2, and/or ERF5; and a second promoter operably-linked to cDNA encoding an enzyme involved in nicotine biosynthesis. In another embodiment, a plant cell line comprises an expression vector comprising a first promoter operably-linked to cDNA encoding Myc3, Myc4, ERF2, and/or ERF5; and a second promoter operably-linked to cDNA encoding an enzyme involved in nicotine biosynthesis. In another embodiment, a transgenic plant comprises an expression vector comprising a first promoter operably-linked to cDNA encoding Myc3, Myc4, ERF2, and/or ERF5; and a second promoter operably-linked to cDNA encoding an enzyme involved in nicotine biosynthesis. In a preferred embodiment, the enzyme is PMT involved in nicotine biosynthesis. In another embodiment, methods for genetically modulating the production level of nicotine are provided, comprising: introducing an expression vector comprising a first promoter operably-linked to cDNA encoding Myc3, Myc4, ERF2, and/or ERF5; and a second promoter operably-linked to cDNA encoding an enzyme involved in nicotine biosynthesis. FIG. 11 shows the polynucleotide sequence for NtMyc3 cDNA (SEQ ID NO:1). FIG. 12 shows the polypeptide sequence for NtMyc3 protein (SEQ ID NO:2). FIG. 13 shows the polynucleotide sequence for NtMyc4 cDNA (SEQ ID NO:3). FIG. 14 shows the polypeptide sequence for NtMyc4 protein (SEQ ID NO:4). FIG. 15 shows the polynucleotide sequence for NtERF2 cDNA (SEQ ID NO:5), which encodes for Ethylene-responsive transcription factor 2 (Swiss Protein Accession No. Q40479; Plant Cell 7 (2), p 173-182 (1995; PUBMED #7756828). FIG. 16 shows the polynucleotide sequence for NtERF5 cDNA (SEQ ID NO:6), which encodes ERF Transcription Factor 5 (NCBI Accession No. AY655738; Mol. Plant Microbe Interact. 17 (10), p 1162-1171 (2004); PUBMED #15497409). NtERF5 cDNA (SEQ ID NO:6) also refers to sequence published in Mol. Plant Microbe Interact. 17 (10), p 1162-1171 (2004).

Another embodiment is directed to an isolated cDNA encoding NtMyc3 (SEQ ID NO:1), or fragments thereof. Another embodiment is directed to an isolated cDNA encoding NtMyc3 and having at least 85%, 90%, 95%, 96%, 97%, 98%, and 99% sequence identity to SEQ ID NO:1, or variant fragments thereof.

Another embodiment is directed to an isolated cDNA encoding NtMyc4 (SEQ ID NO:3), or fragments thereof. Another embodiment is directed to an isolated cDNA encoding NtMyc4 and having at least 85%, 90%, 95%, 96%, 97%, 98%, and 99% sequence identity to SEQ ID NO:3, or variant fragments thereof.

Another embodiment is directed to an isolated polypeptide NtMyc3 (SEQ ID NO:2), or fragments thereof. Another embodiment is directed to an isolated polypeptide NtMyc3 and having at least 85%, 90%, 95%, 96%, 97%, 98%, and 99% sequence identity to SEQ ID NO:2, or fragments thereof.

Another embodiment is directed to an isolated polypeptide NtMyc4 (SEQ ID NO:4), or fragments thereof. Another embodiment is directed to an isolated polypeptide NtMyc4 and having at least 85%, 90%, 95%, 96%, 97%, 98%, and 99% sequence identity to SEQ ID NO:4, or variant fragments thereof.

Another embodiment is directed to an expression vector comprising an isolated cDNA encoding NtMyc3 (SEQ ID NO:1), or fragments thereof. Another embodiment is directed to an expression vector comprising an isolated cDNA encoding NtMyc3 and having at least 85%, 90%, 95%, 96%, 97%, 98%, and 99% sequence identity to SEQ ID NO:1, or fragments thereof.

Another embodiment is directed to an expression vector comprising an isolated cDNA encoding NtMyc4 (SEQ ID NO:3), or fragments thereof. Another embodiment is directed to an expression vector comprising an isolated cDNA encoding NtMyc4 and having at least 85%, 90%, 95%, 96%, 97%, 98%, and 99% sequence identity to SEQ ID NO:3, or fragments thereof.

Another embodiment is directed to an expression vector comprising an isolated cDNA encoding NtMyc3 and having at least 85%, 90%, 95%, 96%, 97%, 98%, and 99% sequence identity to SEQ ID NO:1, or fragments thereof; and an isolated cDNA encoding NtMyc4 and having at least 85%, 90%, 95%, 96%, 97%, 98%, and 99% sequence identity to SEQ ID NO: 3, or fragments thereof.

Another embodiment is directed to an expression vector comprising cDNA encoding a Myc transcriptional factor and/or cDNA encoding a ERF transcriptional factor. In particular, this embodiment is directed to an expression vector comprising: a first sequence comprising an isolated cDNA encoding NtMyc3 and having at least 85%, 90%, 95%, 96%, 97%, 98%, and 99% sequence identity to SEQ ID NO:1, or fragments thereof; and/or a second sequence comprising an isolated cDNA encoding NtMyc4 and having at least 85%, 90%, 95%, 96%, 97%, 98%, and 99% sequence identity to SEQ ID NO:3, or fragments thereof; and an isolated cDNA encoding NtERF2 and having at least 85%, 90%, 95%, 96%, 97%, 98%, and 99% sequence identity to SEQ ID NO:5, or fragments thereof; and/or an isolated cDNA encoding NtERF5 and having at least 85%, 90%, 95%, 96%, 97%, 98%, and 99% sequence identity to SEQ ID NO:6, or fragments thereof, wherein the SEQ ID NO:5 represents the cDNA sequence encoding NtERF2, and the SEQ ID NO:6 represents the cDNA sequence encoding NtERF5.

Another embodiment is directed to a plant cell line comprising an expression vector comprising an isolated cDNA encoding NtMyc3 and having at least 85%, 90%, 95%, 96%, 97%, 98%, and 99% sequence identity to SEQ ID NO:1, or fragments thereof; and/or an isolated cDNA encoding NtMyc4 and having at least 85%, 90%, 95%, 96%, 97%, 98%, and 99% sequence identity to SEQ ID NO:3, or fragments thereof.

Another embodiment is directed to a plant cell line comprising an expression vector comprising at least one ERF transcriptional factor and at least one Myc transcriptional factor. In particular, this embodiment is directed to a plant cell line comprising an expression vector comprising an isolated cDNA encoding NtMyc3 and having at least 85%, 90%, 95%, 96%, 97%, 98%, and 99% sequence identity to SEQ ID NO:1, or fragments thereof; and/or an isolated cDNA encoding NtMyc4 and having at least 85%, 90%, 95%, 96%, 97%, 98%, and 99% sequence identity to SEQ ID NO:3, or fragments thereof; and an isolated cDNA encoding NtERF2 and having at least 85%, 90%, 95%, 96%, 97%, 98%, and 99% sequence identity to SEQ ID NO: 5, or fragments thereof; and/or an isolated cDNA encoding NtERF5 and having at least 85%, 90%, 95%, 96%, 97%, 98%, and 99% sequence identity to SEQ ID NO:6, or fragments thereof, wherein the SEQ ID NO:5 represents the cDNA sequence encoding NtERF2, and the SEQ ID NO:6 represents the cDNA sequence encoding NtERF5.

Another embodiment is directed to a transgenic plant comprising an expression vector comprising an isolated cDNA encoding NtMyc3 and having at least 85%, 90%, 95%, 96%, 97%, 98%, and 99% sequence identity to SEQ ID NO:1, or fragments thereof; and/or an isolated cDNA encoding NtMyc4 and having at least 85%, 90%, 95%, 96%, 97%, 98%, and 99% sequence identity to SEQ ID NO:3, or fragments thereof. In a preferred embodiment, the transgenic plant is a tobacco plant.

Another embodiment is directed to a transgenic plant comprising an expression vector comprising at least one Myc transcriptional factor and at least one ERF transcriptional factor. Another embodiment is directed to a transgenic plant comprising an expression vector comprising an isolated cDNA encoding NtMyc3 and having at least 85%, 90%, 95%, 96%, 97%, 98%, and 99% sequence identity to SEQ ID NO:1, or fragments thereof; and/or an isolated cDNA encoding NtMyc4 and having at least 85%, 90%, 95%, 96%, 97%, 98%, and 99% sequence identity to SEQ ID NO:3, or fragments thereof; and an isolated cDNA encoding NtERF2 and having at least 85%, 90%, 95%, 96%, 97%, 98%, and 99% sequence identity to SEQ ID NO: 5, or fragments thereof; and/or an isolated cDNA encoding NtERF5 and having at least 85%, 90%, 95%, 96%, 97%, 98%, and 99% sequence identity to SEQ ID NO:6, or fragments thereof, wherein the SEQ ID NO:5 represents the cDNA sequence encoding NtERF2, and the SEQ ID NO:6 represents the cDNA sequence encoding NtERF5. In a preferred embodiment, the transgenic plant is a tobacco plant.

Another embodiment is directed to a method for genetically regulating nicotine levels in plants, comprising introducing into a plant an expression vector comprising an isolated cDNA encoding NtMyc3 and having at least 85%, 90%, 95%, 96%, 97%, 98%, and 99% sequence identity to SEQ ID NO:1, or fragments thereof; and/or an isolated cDNA encoding NtMyc4 and having at least 85%, 90%, 95%, 96%, 97%, 98%, and 99% sequence identity to SEQ ID NO:3, or fragments thereof. In a preferred embodiment, the transgenic plant is a tobacco plant.

Another embodiment is directed to a method for genetically regulating nicotine levels in plants, comprising introducing into a plant an expression vector comprising an isolated cDNA encoding NtMyc3 and having at least 85%, 90%, 95%, 96%, 97%, 98%, and 99% sequence identity to SEQ ID NO:1, or fragments thereof; and/or an isolated cDNA encoding NtMyc4 and having at least 85%, 90%, 95%, 96%, 97%, 98%, and 99% sequence identity to SEQ ID NO:3, or fragments thereof; and an isolated cDNA encoding NtERF2 and having at least 85%, 90%, 95%, 96%, 97%, 98%, and 99% sequence identity to SEQ ID NO:5, or fragments thereof; and/or an isolated cDNA encoding NtERF5 and having at least 85%, 90%, 95%, 96%, 97%, 98%, and 99% sequence identity to SEQ ID NO:6, or fragments thereof, wherein the SEQ ID NO:5 represents the cDNA sequence encoding NtERF2, and the SEQ ID NO:6 represents the cDNA sequence encoding NtERF5. In a preferred embodiment, the transgenic plant is a tobacco plant. In a preferred embodiment, the nicotine level in the genetically-modified plant is increased. In another preferred embodiment, the nicotine level in the genetically-modified plant is decreased.

Various embodiments are directed to seeds derived from genetically-modified transgenic plants described herein.

Various embodiments are directed to various polynucleotide molecules that can suppress the expression levels of genes involved in the biosynthetic pathways for the production of alkaloids, flavonoids, and nicotine. Examples of suitable compositions include ERF and Myc anti-sense polynucleotides that are complementary to ERF and Myc transcript sequences, such as RNAi molecules, microRNAs, and other dominant negative constructs known to persons skilled in the art.

It will be appreciated that, although specific embodiments of the invention have been described herein for purposes of illustration, various modifications may be made without departing from the spirit and scope of the invention. Accordingly, the invention is not limited except as by the appended claims.

1.-21. (canceled)

22. An expression vector comprising a heterologous promoter operably linked to a nucleic acid molecule comprising a nucleic acid sequence at least 95% identical to SEQ ID NO: 1.

23. The expression vector of claim 22, wherein the heterologous promoter is a constitutively active promoter.

24. The expression vector of claim 22, wherein the heterologous promoter is an inducible promoter.

25. The expression vector of claim 22, wherein the nucleic acid molecule comprises a nucleic acid sequence at least 96% identical to SEQ ID NO: 1.

26. The expression vector of claim 22, wherein the nucleic acid molecule comprises a nucleic acid sequence at least 97% identical to SEQ ID NO: 1.

27. The expression vector of claim 22, wherein the nucleic acid molecule comprises a nucleic acid sequence at least 98% identical to SEQ ID NO: 1.

28. The expression vector of claim 22, wherein the nucleic acid molecule comprises a nucleic acid sequence at least 99% identical to SEQ ID NO: 1.

29. The expression vector of claim 22, wherein the nucleic acid molecule comprises a nucleic acid sequence 100% identical to SEQ ID NO: 1.

30. The expression vector of claim 22, wherein the nucleic acid molecule encodes the amino acid sequence of SEQ ID NO: 2.

31. A tobacco plant or tobacco seed comprising an expression vector comprising a heterologous promoter operably linked to a nucleic acid molecule comprising a nucleic acid sequence at least 95% identical to SEQ ID NO: 1.

32. The tobacco plant or tobacco seed of claim 31, wherein the tobacco plant or tobacco seed is a Nicotiana tabacum plant or a Nicotiana tabacum seed.

33. The tobacco plant or tobacco seed of claim 31, wherein the heterologous promoter is a constitutively active promoter.

34. The tobacco plant or tobacco seed of claim 31, wherein the heterologous promoter is an inducible promoter.

35. The tobacco plant or tobacco seed of claim 31, wherein the nucleic acid molecule comprises a nucleic acid sequence at least 96% identical to SEQ ID NO: 1.

36. The tobacco plant or tobacco seed of claim 31, wherein the nucleic acid molecule comprises a nucleic acid sequence at least 97% identical to SEQ ID NO: 1.

37. The tobacco plant or tobacco seed of claim 31, wherein the nucleic acid molecule comprises a nucleic acid sequence at least 98% identical to SEQ ID NO: 1.

38. The tobacco plant or tobacco seed of claim 31, wherein the nucleic acid molecule comprises a nucleic acid sequence at least 99% identical to SEQ ID NO: 1.

39. The tobacco plant or tobacco seed of claim 31, wherein the nucleic acid molecule comprises a nucleic acid sequence 100% identical to SEQ ID NO: 1.

40. The tobacco plant or tobacco seed of claim 31, wherein the nucleic acid molecule encodes the amino acid sequence of SEQ ID NO: 2.


SEQUENCE LISTING (10 sequences)

SEQ ID NO: 1 - DNA, 2040 bp, Nicotiana tabacum
  source 1..2040: mol_type = other DNA
atgactgattacagcttacccaccatgaatttgtggaatactagtggtactaccgatgacaacgtttctatgatggaatcttttatgtcttctgatctcacttcattttgggctacttctaattctactactgctgctgttacctctaattctaatcttattccagttaataccctaactgttcttcttccgtcttcttgtgcttctactgtcacagctgtggctgtcgatgcttcaaaatccatgtcttttttcaaccaagaaactcttcagcagcgtcttcaaaccctcattgatggtgctcgtgagacgtggacctatgccatcttttggcagtcatccgtcgttgatttatcgagtccgtttgtgttgggctggggagatggttactacaaaggtgaagaagataaagccaataggaaattagctgtttcttctcctgcttatattgctgagcaagaacaccgaaaaaaggttctccgggagctgaattcgttgatctccggcacgcaaaccggcactgatgatgccgtcgatgaagaagttaccgacactgaatggttcttccttatttccatgacccaatcgtttgttaacggaagtgggcttccgggtcaggccttatacaattccagccctatttgggtcgccggagcagagaaattggcagcttcccactgcgaacgggctcggcaggcccagggattcgggcttcagacgatggtttgtattccttcagcaaacggcgtggttgaattgggctccacggagttgataatccagagttgtgatctcatgaacaaggttagagtattgtttaacttcaataatgatttgggctctggttcgtgggctgtgcagcccgagagcgatccgtccgctctttggctcactgatccatcgtctgcagctgtagaagtccaagatttaaatacagttaaggcaaattcagttccatcaagtaatagtagtaagcaagttgtgtttgataatgagaataatggtcacagttctgataatcagcaacagcagcattctaagcatgaaacacaaggatttttcacaagggagttgaatttttcagaatttgggtttgatggaagtagtaataataggaatgggaattcatcactttcttgcaagccagagtcgggggaaatcttgaattttggtgatagtactaagaaaagtgcaaatgggaacttattttcgggtcagtcccattttggggcaggggaggagaataagaacaagaaaaggtcacctgcttccagaggaagcaatgaagaaggaatgctttcatttgtttcgggtacaatcttgcctgcagcttctggtgcgatgaagtcaagtggaggtgtaggtgaagactctgatcattcggatcttgaggcctcagtggtgaaagaagctgaaagtagtagagttgtagaacccgaaaagaggccaaagaagcgaggaaggaagccagcaaatggacgggaggaacctttgaatcacgtcgaagcagagaggcaaaggagagagaaattaaaccaaaggttctacgcattaagagctgttgttccgaatgtgtccaagatggacaaggcatcactgcttggagatgcaatttcatatattaatgagctgaagttgaagcttcaaaatacagaaacagatagagaagaattgaagagccaaatagaagatttaaagaaagaattagttagtaaagactcaaggcgccctggtcctccaccatcaaatcatgatcacaagatgtctagccatactggaagcaagattgtagacgtggatatagatgttaagataattggatgggatgcgatgattcgtatacaatgtaataaaaagaatcatccagctgcaaggttaatggtagccctcaaggagttagatctagatgtgcaccatgccagtgtttcagtggtgaacgatttgatgatccaacaagccactgtgaaaatgggtagcagactttacacggaagagcaacttaggatagcattgacatccagagttgctgaaacacgctaa

SEQ ID NO: 2 - protein, 679 aa, Nicotiana tabacum
MTDYSLPTMNLWNTSGTTDDNVSMMESFMSSDLTSFWATSNSTTAAVTSNSNLIPVNTLTVLLPSSCASTVTAVAVDASKSMSFFNQETLQQRLQTLIDGARETWTYAIFWQSSVVDLSSPFVLGWGDGYYKGEEDKANRKLAVSSPAYIAEQEHRKKVLRELNSLISGTQTGTDDAVDEEVTDTEWFFLISMTQSFVNGSGLPGQALYNSSPIWVAGAEKLAASHCERARQAQGFGLQTMVCIPSANGVVELGSTELIIQSCDLMNKVRVLFNFNNDLGSGSWAVQPESDPSALWLTDPSSAAVEVQDLNTVKANSVPSSNSSKQVVFDNENNGHSSDNQQQQHSKHETQGFFTRELNFSEFGFDGSSNNRNGNSSLSCKPESGEILNFGDSTKKSANGNLFSGQSHFGAGEENKNKKRSPASRGSNEEGMLSFVSGTILPAASGAMKSSGGVGEDSDHSDLEASVVKEAESSRVVEPEKRPKKRGRKPANGREEPLNHVEAERQRREKLNQRFYALRAVVPNVSKMDKASLLGDAISYINELKLKLQNTETDREELKSQIEDLKKELVSKDSRRPGPPPSNHDHKMSSHTGSKIVDVDIDVKIIGWDAMIRIQCNKKNHPAARLMVALKELDLDVHHASVSVVNDLMIQQATVKMGSRLYTEEQLRIALTSRVAETR

SEQ ID NO: 3 - DNA, 2046 bp, Nicotiana tabacum
  source 1..2046: mol_type = other DNA
atgactgattacagcttacccaccatgaatttgtggaatactagtggtactaccgatgacaacgttactatgatggaagcttttatgtcttctgatctcacttcattttgggctacttctaattctactgctgttgctgctgttacctctaattctaatcatattccagttaataccccaacggttcttcttccgtcttcttgtgcctctactgtcacagctgtggctgtcgatgcttcaaaatccatgtcttttttcaaccaagaaacccttcaacagcgtcttcaaacgctcattgatggtgctcgtgagacgtggacctatgccatcttttggcagtcatccgccgttgatttaacgagtccgtttgtgttgggctggggagatggttactacaaaggtgaagaagataaagccaataggaaattagctgtttcttctcctgcttatatagctgagcaagaacaccggaaaaaggttctccgggagctgaattcgttgatttccggcacgcaaaccggcactgatgatgccgtcgatgaagaagttaccgacactgaatggttcttccttatttccatgacccagtcgtttgttaacggaagtgggcttccgggtcaggccttatacaattccagccctatttgggtcgccggagcagagaaattggcagcttcccactgcgaacgggctcggcaggcccagggattcgggcttcagacgatggtttgtattccttcagcaaacggcgtggttgaattgggctccacggagttgattattcagagttctgatctcatgaacaaggttagagtattgtttaacttcaataatgatttgggctctggttcgtgggctgtgcaacccgagagcgatccgtccgctctttggctcactgatccatcgtctgcagctgtacaagtcaaagatttaaatacagttgaggcaaattcagttccatcaagtaatagtagtaagcaagttgtatttgataatgagaataatggtcacagttgtgataatcagcaacagcaccattctcggcaacaaacacaaggattttttacaagggagttgaacttttcagaattcgggtttgatggaagtagtaataataggaatgggaattcatcactttcttgcaagccagagtcgggggaaatcttgaattttggtgatagcactaagaaaagtgcaaatgggaacttattttccggtcagtctcattttggtgcaggggaggagaataagaagaagaaaaggtcacctgcttccagaggaagcaatgaagaaggaatgctttcatttgtttcaggtacaatcttgcctgcagcttctggtgcgatgaagtcaagtggatgtgtcggtgaagactcctctgatcattcggatcttgaggcctcagtggtgaaagaagctgaaagtagtagagttgtagaacccgaaaagaggccaaagaagcgaggaaggaagccagcaaatggacgtgaggaacctttgaatcacgtcgaagcagagaggcaaaggagagagaaattaaaccaaaggttctacgctttaagagctgttgttccgaatgtgtccaagatggacaaggcatcactgcttggagatgcaatttcatatattaatgagctgaagttgaagcttcaaactacagaaacagatagagaagacttgaagagccaaatagaagatttgaagaaagaattagatagtaaagactcaaggcgccctggtcctccaccaccaaatcaagatcacaagatgtctagccatactggaagcaagattgtagatgtggatatagatgttaagataattggatgggatgcgatgattcgtatacaatgtaataaaaagaaccatccagctgcaaggttaatggtagccctcaaggagttagatctagatgtgcaccatgccagtgtttcagtggtgaatgatttgatgatccaacaagccacagtgaaaatgggtagcagactttacacggaagagcaacttaggatagcattgacatccagagttgctgaaacacgctaa

SEQ ID NO: 4 - protein, 681 aa, Nicotiana tabacum
MTDYSLPTMNLWNTSGTTDDNVTMMEAFMSSDLTSFWATSNSTAVAAVTSNSNHIPVNTPTVLLPSSCASTVTAVAVDASKSMSFFNQETLQQRLQTLIDGARETWTYAIFWQSSAVDLTSPFVLGWGDGYYKGEEDKANRKLAVSSPAYIAEQEHRKKVLRELNSLISGTQTGTDDAVDEEVTDTEWFFLISMTQSFVNGSGLPGQALYNSSPIWVAGAEKLAASHCERARQAQGFGLQTMVCIPSANGVVELGSTELIIQSSDLMNKVRVLFNFNNDLGSGSWAVQPESDPSALWLTDPSSAAVQVKDLNTVEANSVPSSNSSKQVVFDNENNGHSCDNQQQHHSRQQTQGFFTRELNFSEFGFDGSSNNRNGNSSLSCKPESGEILNFGDSTKKSANGNLFSGQSHFGAGEENKKKKRSPASRGSNEEGMLSFVSGTILPAASGAMKSSGCVGEDSSDHSDLEASVVKEAESSRVVEPEKRPKKRGRKPANGREEPLNHVEAERQRREKLNQRFYALRAVVPNVSKMDKASLLGDAISYINELKLKLQTTETDREDLKSQIEDLKKELDSKDSRRPGPPPPNQDHKMSSHTGSKIVDVDIDVKIIGWDAMIRIQCNKKNHPAARLMVALKELDLDVHHASVSVVNDLMIQQATVKMGSRLYTEEQLRIALTSRVAETR

SEQ ID NO: 5 - DNA, 702 bp, synthetic construct
  misc_feature 1..702: note = cDNA sequence of NtERF2
  source 1..702: mol_type = other DNA
atgtatcaaccaatttcgaccgagctacctccgacgagtttcagtagtctcatgccatgtttgacggatacatggggtgacttgccgttaaaagttgatgattccgaagatatggtaatttatgggctcttaagtgacgctttaactgccggatggacgccgtttaatttaacgtccaccgaaataaaagccgagccgagggaggagattgagccagctacgattcctgttccttcagtggctccacctgcggagactacgacggctcaagccgttgttcccaaggggaggcattataggggcgttaggcaaaggccgtgggggaaatttgcggcggaaataagggacccagctaaaaacggcgcacgggtttggctagggacttatgagacggctgaagaagccgcgctcgcttatgataaagcagcttacaggatgcgcggctccaaggctctattgaattttccgcataggatcggcttaaatgagcctgaaccggttagactaaccgctaagagacgatcacctgaaccggctagctcgtcaatatcatcggctttggaaaatggctcgccgaaacggaggagaaaagctgtagcggctaagaaggctgaattagaagtgcaaagccgatcaaatgctatgcaagttgggtgccagatggaacaatttccagttggcgagcagctattagtcagttaa

SEQ ID NO: 6 - DNA, 705 bp, synthetic construct
  misc_feature 1..705: note = NtERF5 cDNA
  source 1..705: mol_type = other DNA
atgtcaagtaactcaagcccactagaaatagacacttcattttcacattccaacttcttctttctccaagatcaatcaccaattttacaatgggatgatgatcttttcttcaatgatccatggtttgatgatgatcaatcaccaattataccatgtaactcagagaaagatgaaaatcatcaagtatttgaagaatcctcagacaatacaatcatgtcaaaaggaagtagccatggtcaagaattagaagaggtaacatcccaagaagaaaaagaaaaagaagaagaagaaaaacactatataggagttagaaaaaggccatggggtaaatatgcagcagaaataagagattcaacaagaaatggaattagggtttggttagggacatttgatacagctgaagaagctgctttagcttatgatcaagctgcattatcgatgagaggtccttggtctcttcttaattttccattggagaaagtcaagaaatcacttgaaaaaattgagtattcttgtaaagatggattgtctcctgctgctgttctaaaagctactcataaaacaaggagagtgaagcataaaagaagtagtagaaagaaaaagaataaagaaactcataatgttattgtttttgaggacttgggtgctgagttattagaagagcttttaatgacttcatcacaacattcgtgtcgaagggactga

SEQ ID NO: 7 - DNA, 57 bp, synthetic construct
  misc_feature 1..57: note = NtPMT1a promoter
  source 1..57: mol_type = other DNA
ctgcacgttgtaatgaatttttaactattatattatatcgagttgcgccctccactc

SEQ ID NO: 8 - DNA, 57 bp, synthetic construct
  misc_feature 1..57: note = Synthesized
  source 1..57: mol_type = other DNA
cttcttttttatatctaaaataaagataataaaatattcgattttctccctccactc

SEQ ID NO: 9 - DNA, 14 bp, synthetic construct
  misc_feature 1..14: note = G-Box
  source 1..14: mol_type = other DNA
ctgcacgttgtaat

SEQ ID NO: 10 - DNA, 14 bp, synthetic construct
  misc_feature 1..14: note = Mutant G-Box
  source 1..14: mol_type = other DNA
ctgcttaatgtaat